(19) United States
(12) Patent Application Publication
(10) Pub. No.: US 2007/0009454 A1
(43) Pub. Date: Jan. 11, 2007

(54) Make-up method involving a magnetic interaction

(75) Inventor: Ludovic Thevenet, Bourg La Reine (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/482,165

(22) Filed: Jul. 7, 2006

Related U.S. Application Data
(60) Provisional application No. 60/700,312, filed on Jul. 19, 2005.

(30) Foreign Application Priority Data
Jul. 8, 2005 (FR) 05 52125

(51) Int. Cl. A61K 8/19

(52) U.S. Cl. 424/61; 424/70.7

(57) ABSTRACT

A method of making up or decorating keratinous materials includes applying, to the keratinous materials, a first layer of a first composition or a first structure having magnetic properties. The method includes applying, to at least a portion of the first layer or first structure, a second layer of a second composition or a second structure interacting magnetically with the first layer or with the first structure. At least one of the first and second structures includes dispersed magnetic bodies in a non-magnetic medium. A kit may include the first composition or the first structure and the second composition or the second structure.

This non-provisional application claims the benefit of French Application No. 05 52125, filed on Jul. 8, 2005, and U.S. Provisional Application No. 60/700,312, filed on Jul. 19, 2005, the entire disclosures of which are incorporated herein by reference.

The present invention relates to a method of making up a surface, natural such as the skin, nails, hair or lips, or artificial such as false nails, and to various kits, articles and compositions for implementing the method.

### BACKGROUND

Make-up methods that successively apply two compositions are known. The second composition applied to the first composition is aimed, for example, at improving retention properties or gloss.

### SUMMARY

Exemplary embodiments of the present invention may, for example, allow a makeup to be removed or modified readily and/or to create novel esthetic effects.

In exemplary embodiments, a method of making up and/or decorating keratinous materials, in particular the skin, nails, or eyelashes, may include applying, to the keratinous materials, a first layer of a first composition or a first structure presenting magnetic properties; and applying, to at least a portion of the first layer or first structure, a second layer of a second composition or a second structure interacting magnetically with the first layer or with the first structure. At least one of the first and second structures may include dispersed magnetic bodies in a non-magnetic medium.

The term “makeup” should be construed in its broad sense and may encompass applying or forming a decoration to or on the body or face.

The term “dispersed” means that the composition or structure is “distributed” in a non-magnetic medium. When the composition or structure is intended for direct application to keratinous materials, the medium may be physiologically acceptable. A monolithic magnet produced by sintering may not be an example of dispersed magnetic particles since the magnetic particles have been agglomerated.

“Magnetic properties” means a non-zero magnetic susceptibility, sufficient for the effects due to the magnetic interaction, for example visual and/or of attraction, to be perceived by the user during the implementation of the method.

“Magnetic interaction” means an interaction between at least one magnetic body, i.e., a body that is magnetised and/or magnetisable, and another body generating a magnetic field. Such an interaction may generate attraction or repulsion forces and/or cause at least one body to change orientation and/or to move relative to another body.

The magnetic interaction due to, for example, permanent magnetization of the first layer or first structure and/or the second layer or second structure may ensure that the second layer or structure is held reversibly on the first layer or structure. In a variation, this retention may be at least partially ensured in a manner other than by magnetic interaction, for example, by adhesion using an adhesive. The adhesive may belong to the second layer or structure, for example.

At least one of the first and second layers or structures may be permanently magnetized.

The second layer or structure may be a second layer of a composition including dispersed magnetic bodies and the magnetic interaction may contribute to orientating and/or displacing at least some of the magnetic bodies present in the second layer in order, for example, to create a novel esthetic effect, in particular the appearance of a motif. The motif may have a form that may be linked to that of the first layer or structure and/or to the disposition of magnetic field lines.

In various exemplary embodiments, a first layer of composition may be applied and a second structure may be caused to cover at least a portion of the first layer of composition. The first layer of composition may contain a volatile solvent and the second structure may be deposited on the first layer after it has dried. The second structure may be, for example, a false nail, a false tattoo, a beauty spot, a jewel, a film, or patch, or any other decorative article, in particular powder, flakes, or fibers. The second structure may be flexible and, if appropriate, may be cut to a desired shape or in accordance with a given motif. The magnetic interaction between the first layer of composition and the second structure may contribute to maintaining the second structure on the first layer while allowing the user to readily remove or replace the second structure, advantageously without damaging the first layer.

When the first layer is applied to a nail, the nail may advantageously have the appearance of a varnished nail. The magnetic properties of the first layer allow the user to readily attach a decorative article, for example, a false nail, to the nail and then to change it, for example, to match clothing.

The removable nature of the false nails or other articles also allows the user to remove them temporarily to carry out certain tasks, then to replace them without risking a loss of adhesion.

In various exemplary embodiments, a first structure may be applied, and a second layer of composition or structure may be caused to cover at least a portion of the first structure. The first structure may thus be adhesive, for example. The first structure may include a flexible film which may be adhesive and/or may be cut to shape or in accordance with a desired motif. The first structure may also be deposited by transfer. When a second layer of composition is applied to the structure, the magnetic interaction may, for example, modify the orientation of magnetic pigments contained in the second layer to create an esthetic effect. The magnetic field lines may be distributed to allow motifs to appear, which are difficult to produce in other ways.

In various exemplary embodiments, a first layer of composition having magnetic properties may be applied as well as a second layer of composition including magnetic bodies that may be orientated with the first layer of composition under the effect of a magnetic field.

The first layer of composition may be applied in accordance with a motif, for example manually or using an inkjet printer or in a different manner, for example, by transfer.

In all of the above examples, the first layer or structure may be permanently magnetized, in order, for example, to interact with at least one magnetic body of the second layer or structure, which need not be permanently magnetized but which may have non-zero magnetic susceptibility. The second layer or structure may still be permanently magnetized, which may increase the magnetic interaction.

A permanently magnetized layer or structure may interact magnetically with at least one pigment having a non-zero magnetic susceptibility contained in the other layer. The magnetic interaction may cause a motif to be formed in the layer including the magnetic pigment following a change in the orientation of particles of the pigment as they align with the field lines.

Various exemplary embodiments provide a hand makeup kit including at least one and preferably at least five articles having magnetic properties and a form that is suitable for fixing them to the nails by magnetic attraction. The article or articles may be, for example, false nails.

Various embodiments may also provide a magnetic false nail. The false nail may include a magnet and/or permanently magnetized dispersed magnetic particles.

Various exemplary embodiments provide a makeup kit that may include at least one receptacle containing a composition having magnetic properties and at least one magnetic structure capable of being fixed by magnetic attraction to a surface on which a layer of the composition contained in the receptacle has been deposited.

The composition having magnetic properties may include, for example, a dispersion of magnetic bodies, such as particles, for example, in particular pigments, having non-zero magnetic susceptibility.

The magnetic structure may be a false nail, a jewel, powder, a beauty spot, a flake, or a false tattoo, including, among other things, a permanent magnet or a dispersion of bodies, in particular magnetic particles and/or fibers, which are permanently magnetized.

Various exemplary embodiments may also provide a makeup kit including a first receptacle containing a first composition containing bodies including metallic iron, in particular soft iron and a second receptacle containing a second composition including magnetic bodies, the second composition being capable, when it is applied in the form of a layer above or below a layer formed by the first composition, of interacting magnetically with the first composition. The magnetic bodies of at least one of the first and second compositions may be permanently magnetized.

### BRIEF DESCRIPTION OF THE DRAWINGS

Various details of the present invention may be better understood on reading the following detailed description of non-limiting exemplary embodiments, and on examining the accompanying drawings, which form an integral part of the description, and in which:

FIG. 1 illustrates the application of a first layer of composition to a nail;

FIG. 2 illustrates positioning a magnetic false nail on the deposited layer of composition;

FIG. 3 is a diagrammatic representation of an exemplary kit for implementing the method shown in FIGS. 1 and 2;

FIGS. 4 and 5 are two cross sections of a false nail in accordance with various exemplary embodiments of the invention;

FIGS. 6 to 9 show exemplary embodiments of the invention;

FIGS. 10 and 14 show exemplary kits;

FIGS. 11 and 12 show the use of the exemplary kit of FIG. 10; and

FIG. 13 is a section on line XIII-XIII of FIG. 11.

### DETAILED DESCRIPTION OF EMBODIMENTS

FIG. 1 shows a first layer 1 of a composition P including dispersed magnetic bodies being applied to a nail.

The first layer 1 of composition P is, for example, applied to the nail using an applicator 2, such as a brush, which brush may form part of a packaging and applicator device 3 of a kit 4 shown in FIG. 3.

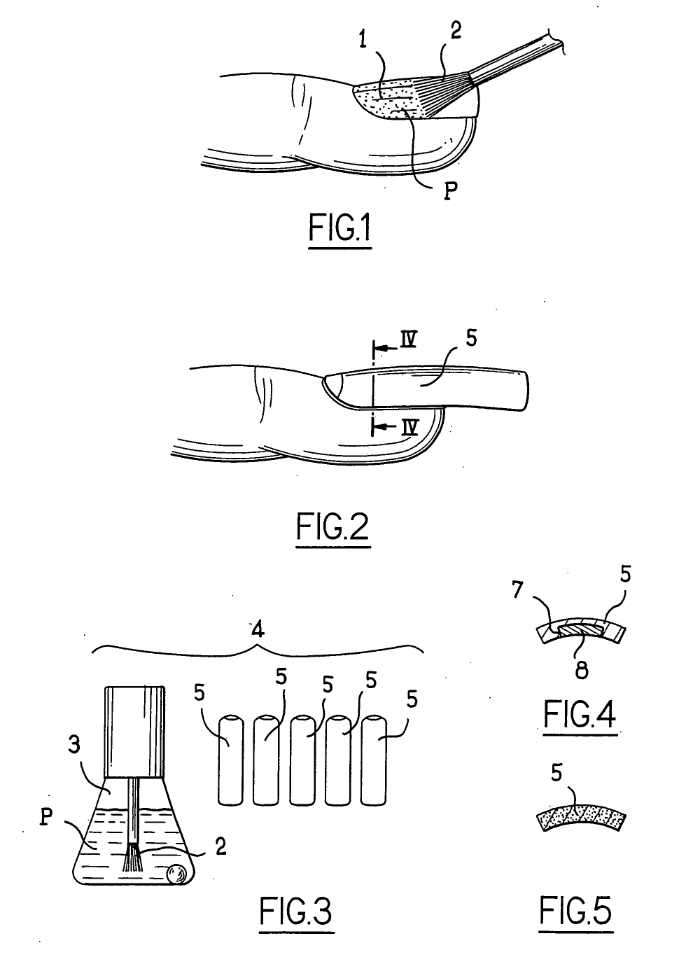

Once the first layer 1 is dry, the user can apply a magnetic structure to the nail, such as a false nail 5, as shown in FIG. 2. The false nail 5 may be arranged to interact magnetically with the first layer 1 and may include a permanent magnet 8, for example, as can be seen in FIG. 4. The magnet 8 may be, for example, secured in a corresponding recess 7 of the false nail 5. The body of the false nail may be molded from a synthetic material, for example, a thermoplastic material. The magnet 8 may be, for example, bonded into the recess 7.

In a variation (not shown), the thermoplastic material may be over molded onto the magnet.

The magnetic field generated by the magnet 8 may be, for example, about 100 mT [millitorr] or more.

As shown in FIG. 5, the false nail 5 may also have magnetic properties because a filler of magnetic particles capable of being permanently magnetized has been incorporated into the thermoplastic synthetic material from which it has been produced.

A plurality of false nails 5, for example, intended to be applied to all the fingers of one hand, may be offered to the user in kit 4, as shown in FIG. 3.

The dispersed magnetic bodies in composition P may include any magnetic material having a non-zero magnetic sensitivity, the material being selected, for example, from nickel, cobalt, iron, their alloys and oxides, in particular Fe₃O₄, and also gadolinium, terbium, dysprosium, erbium, Cu₂MnAl, MnBi, and their alloys and oxides. The magnetic material may be “soft” or “hard” in type. Composition P may contain a ferrofluid.

Composition P may act solely to allow magnetic interaction with the magnetic structure attached to it, such as a false nail.

Composition P may also have an appearance allowing it to be used, if appropriate, without the associated magnetic structure.

Composition P may thus advantageously include at least one coloring agent, which may or may not be magnetic. If appropriate, the coloring agent may produce the magnetic properties of the composition by itself.

The dispersed magnetic bodies in the composition may or may not have a multi-layered structure, including at least one layer of a magnetic material, such as iron, nickel, cobalt, their alloys and oxides, and in particular Fe₃O₄. The magnetic bodies may be coated, and only the core may be magnetic. The shell may constitute, for example, a barrier intended to prevent oxidation, to render it safe, and/or to color it.

The coating may be, for example, an inorganic or an organic material, in particular a polymeric material.

The quantity of magnetic bodies may be sufficient to obtain the desired magnetic interaction. The concentration of magnetic bodies in the composition P may be, for example, in the range from about 0.05% to about 97% by weight, in particular in the range from about 0.1% to about 95% by weight, or preferably in the range from about 0.1% to about 90% by weight, for example, of the order of 3% by weight.

The dimension of the magnetic bodies contained in the composition P may be, for example, in the range of about 1 nm [nanometers] to about 700 μm [micrometers], preferably in the range of about 1 μm to about 500 μm, more preferably in the range of about 10 μm to about 150 μm. The term “dimension” means the dimension given by the statistical grain size distribution at half of the population, termed D50. A relatively small dimension may facilitate ease of application.

The magnetic bodies contained in the composition P may include magnetic pigments, as mentioned above.

Magnetic pigments may include nacres including iron oxide Fe3O4. Pigments having magnetic properties may be, for example, those sold under the trade names COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289) and COLORONA PATINA GOLD (117288) from MERCK or FLAMENCO TWILIGHT RED, FLAMENCO TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424 CB, CHROMA-LITE BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB) and CLOISONNE NU ANTIQUE COPPER (340 XB) from ENGELHARD.

Black iron oxide particles may also be mentioned, for example, those sold under the trade name SICOVIT black E172 by BASF or particles based on acicular soft iron.

Composition P may include at least one special effect coloring agent, for example a goniochromatic coloring agent and/or at least one diffracting pigment.

In one example, for application to a nail, composition P may have the following formulation; the proportions are expressed as a % by weight.

| | |
|---|---|
| Nitrocellulose | 11 |
| N-ethyl-o,p-toluenesulfonamide | 5 |
| Alkyd resin | 10 |
| Isopropanol | 4 |
| Magnetic pigments** | 1.5 |
| Butyl acetate/ethyl acetate, 50/50 | Qsp 100 |

**Pigments based on acicular soft iron.

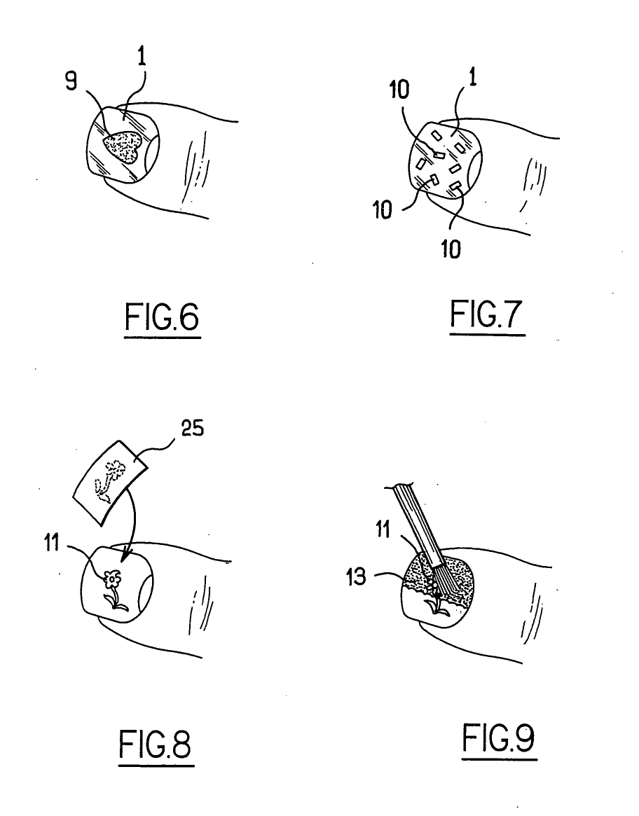

The structure applied to the nail may be other than a false nail and, for example, it may be in the form of a decorative article 9 as shown in FIG. 6, the article possibly being flexible or rigid.

Article 9 may be, for example a film, a patch, or a false tattoo.

Article 9 may be, for example, cut from a film including a filler of permanently magnetized dispersed magnetic particles. In a variation, article 9 may be produced by molding a plastics material and may include a recess receiving a permanent magnet.

Article 9 may include a natural or synthetic stone and may form a jewel.

Article 9 may also include printing or a decorative coating such as, for example, a colored varnish.

FIG. 7 shows the possibility of applying a plurality of decorative elements 10 such as fibers, flakes, or powder to the first layer of composition P. The decorative elements 10 may be permanently magnetized. In a variation, the permanent magnetism may derive from the first layer 1 of the composition P. If appropriate, a protective varnish may be applied to the decorated surface. Under such circumstances, the protective varnish may improve the retention of the element or elements 10 deposited on the nail. The magnetic interaction between the first layer and the decorative elements may contribute to preventing them from moving before and during application of the varnish, allowing the user to position them in the desired configuration before applying the protective varnish, without risking displacing them during application of the layer of varnish.

A first layer of a composition including dispersed magnetic bodies may be applied, if appropriate, as shown in FIG. 8, to form a motif 11 on the surface to be made up. The application may be made manually, for example, using a brush, possibly through a stencil, or using a machine, for example an inkjet printer such as that described, for example in U.S. Pat. No. 5,931,166 (which is hereby incorporated by reference in its entirety), or by transfer, as shown. Motif 11 may be transferred in the form of a film. Motif 11 may be present on a backing strip 25 prior to transfer.

Next, a second layer 13 of a composition also including dispersed magnetic bodies, capable of interacting magnetically with those of the first layer may be applied to the surface, to cover the motif 11 and magnetically interact therewith. The composition that forms the second layer 13 may have, for example, the same formulation as composition P described above and, for example, may contain pigments having non-zero magnetic susceptibility. The orientation of the pigments may be modified by the magnetic interaction with the dispersed magnetic bodies present in the motif 11, which may be permanently magnetized.

The second layer 13, for example, may include aspherical magnetic bodies, having an elongate shape. When subjected to a magnetic field generated by the first layer or structure, the bodies may tend to become orientated with their longitudinal axes aligned with the field lines, and may undergo a change in orientation which results in a change in appearance.

The magnetic bodies may also be non homogeneous in appearance, so that a change in orientation induces a change in appearance.

The second layer 13 may include magnetic pigments selected from those mentioned above and/or magnetic fibers.

The composition intended to form the second layer may be offered in kit form with at least one backing strip 25 carrying a motif to be transferred. In a variation, the backing strip 25 alone may be offered to the user.

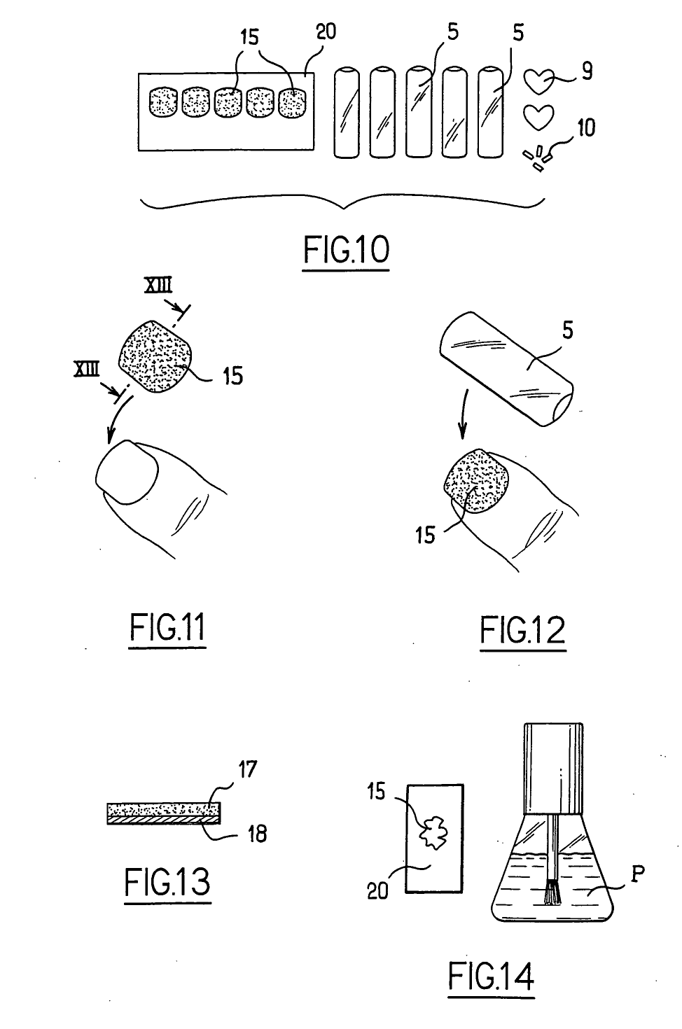

FIG. 10 shows a further makeup kit including at least one first structure 15 having magnetic properties to be applied to the surface to be made up, and at least one second structure, such as a false nail 5 or decorative elements 9 or 10 intended to cover the first structure, and interacting magnetically therewith.

The first structure 15 may be, for example, in the form of a flexible film including, as shown in FIG. 13, a layer of a polymer material 17 including dispersed magnetic bodies and a layer of an adhesive 18 for fixing to the surface to be made up.

In a variation, the film may not have a multi-layered structure but a mono-layered structure, and may have adhesive properties due to, for example, only partial evaporation of a solvent.

The first structure 15 may be, for example, intended to be cut into a shape corresponding to the surface to be made up before or after fixing the first structure 15 to the surface. Once the first structure 15 has been fixed to the surface, the second structure may be applied thereto, as shown in FIG. 12, and may be held thereon by magnetic interaction.

The first structure 15 may be permanently magnetized due to the presence of permanently magnetized magnetic bodies in the polymer layer 17.

The kit of FIG. 10 may include, for example, the same number of first structures 15 as there are fingers to be made up. The first structures 15 may be, for example, fixed to a non-stick surface sheet 20 to allow them to be readily removed for fixing to the surface to be made up.

FIG. 14 shows a further example of a kit in which the first structure 15 may be pre-cut to form a motif. The kit may include a magnetic composition P intended to cover the surface to be made up after application of the first structure thereto, in a manner similar to that described with reference to FIGS. 8 and 9.

The invention is need not be limited to the examples described above. In particular, the invention may be applicable not only to makeup for the nails but also to makeup for the skin, lips, or keratinous fibers.

Composition P, which is intended to cover or be covered, may contain at least one of the following compounds, depending on the nature of the surface that is to be made up.

Goniochromatic Coloring Agents

The term “goniochromatic coloring agent” means a coloring agent which, when the composition is spread over a surface, can produce a color plot in the a*b* plane of the CIE 1976 color space which corresponds to a variation Dh° of the hue angle h° of at least 20° when the angle of observation is varied relative to the normal between 0° and 80°, for an angle of illumination incidence of 45°.

The color plot may be measured, for example, using a spectrogonioreflectometer sold by INSTRUMENT SYSTEMS with reference GON 360 GONIOMETER, after the composition has been spread in the fluid state to a thickness of 300 μm using an automatic spreader onto an ERICHSEN contrast card with reference Typ 24/5, the measurement being carried out on the black background of the card.

The goniochromatic coloring agent may be, for example, selected from interferential multi-layered structures and liquid crystal coloring agents.

In the case of a multi-layered structure, this may, for example, include at least two layers, each layer being carried out, for example, from at least one material selected from the group constituted by the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

The multi-layered structure may or may not be symmetrical with respect to a central layer as regards the chemical nature of the stacked layers. Depending on the thickness and the nature of the various layers, different effects are obtained.

Examples of symmetrical multi-layered interferential structures may include the following structures: Fe₂O₃/SiO₂/Fe₂O₃/SiO₂/Fe₂O₃, a pigment having that structure being sold under the trade name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; pigments having those structures are sold under the trade name XIRONA by MERCK (Darmstadt).

Liquid crystal coloring agents include, for example, silicones or cellulose ethers onto which mesomorphous groups are grafted. Examples of particular goniochromatic liquid crystals which may be used are those sold by CHENIX and those sold under the trade name HELICONE® HC by WACKER.

The goniochromatic coloring agent may also be a nacre, special effect pigment on a synthetic substrate, in particular a substrate of the alumina, silica, borosilicate, iron oxide, or aluminum type or interferential holographic flakes from a polyterephthalate film.

Composition P may also include dispersed goniochromatic fibers. Such fibers may be less than 80 μm in length, for example.

Composition P may also include at least one diffracting pigment, which may if appropriate have magnetic properties.

Diffracting Pigments

The term “diffracting pigment” as used in the present invention means a pigment, which is capable of producing a color variation depending on the angle of observation when illuminated with white light due to the presence of a structure that diffracts light.

A diffracting pigment may include a diffraction matrix, for example, capable of diffracting an incident ray of monochromatic light in predetermined directions.

The diffraction matrix may include a periodic motif, in particular a line, the distance between two adjacent motifs being of the same order of magnitude as the wavelength of the incident light.

When the incident light is polychromatic, the diffraction matrix may separate the various spectral components of the light and may produce a rainbow effect.

Concerning the structure of diffracting pigments, reference should be made to the article “Pigments Exhibiting Diffractive Effects” by Alberto Argoitia and Matt Witzman, 2002, Society of Vacuum Coaters, 45th Annual Technical Conference Proceedings, 2002.

The diffracting pigment may be produced with motifs having different profiles, in particular triangular, symmetrical or unsymmetrical, crenellated, with a constant or non constant width, or even sinusoidal.

The spatial frequency of the matrix and the motif depth may be selected as a function of the desired degree of separation of the various orders. As an example, the frequency may lie in the range of about 500 to about 3000 lines per mm [millimeter].

Preferably, the particles of diffracting pigment each have a flattened form, in particular in the form of a platelet.

The same pigment particle may include two crossed diffraction matrices, which may or may not be perpendicular.

The diffracting pigment may have a multi-layered structure including a layer of reflective material, covered on at least one side by a layer of a dielectric material. This layer may provide the diffracting pigment with better rigidity and durability. The dielectric material may thus, for example, be selected from the following materials: MgF₂, SiO₂, Al₂O₃, AlF₃, CeF₃, LaF₃, NdF₃, SmF₂, BaF₂, CaF₂, LiF and combinations thereof. The reflective material, for example, may be selected from metals and their alloys and also from non-metallic reflective materials. Metals which may be mentioned include Al, Ag, Cu, Au, Pt, Sn, Ti, Pd, Ni, Co, Rd, Nb, Cr and their materials, combinations, or alloys. Such a reflective material may alone constitute the diffracting pigment, which is then a monolayer.

In a variation, the diffracting pigment may include a multi-layered structure including a core of a dielectric material covered on at least one side by a reflective layer, or even completely encapsulating the core. A layer of a dielectric material may also cover the reflective layer or layers. The dielectric material used thus may preferably be inorganic and may, for example, be selected from metal fluorides, metal oxides, metal sulfides, metal nitrides, metal carbides, and combinations thereof. The dielectric material may be in the crystalline, semi-crystalline, or amorphous state. The dielectric material in this configuration, for example, may be selected from the following materials: MgF₂, SiO, SiO₂, Al₂O₃, TiO₂, WO, AlN, BN, B₄C, WC, TiC, TiN, N₄Si₃, ZnS, glass particles, diamond type carbons, and combinations thereof.

In a variation, the diffracting pigment may be composed of a dielectric or preformed ceramic material, such as a mineral in natural lamellae, for example, mica peroskovite or talc, or synthetic lamellae formed from glass, alumina, SiO₂, carbon, an iron oxide/mica, mica coated with BN, BC, graphite, bismuth oxychloride, and combinations thereof.

Instead of a layer of a dielectric material, other materials that improve the mechanical properties may be suitable. Such materials may include silicone, metal silicides, semiconductor materials formed from elements from groups III, IV, and V, metals having a body-centered cubic crystal structure, Cermet compositions or materials, semiconductor glasses, and their various combinations.

In particular, the diffracting pigment used may be selected from those described in United States patent application US 2003/0031870, published on Feb. 13, 2003, the disclosure of which is incorporated herein by reference in its entirety.

A diffracting pigment may include, for example, the following structure: MgF₂/Al/MgF₂, a diffracting pigment having that structure being sold under the trade name SPECTRAFLAIR 1400 Pigment Silver by FLEX PRODUCTS, or SPECTRAFLAIR 1400 Pigment Silver FG. The proportion by weight of MgF₂ may be in the range of about 80% to about 95% of the total weight of the pigment.

The quantity of diffracting pigment may vary, for example, from about 0.1% to about 5% by weight, with respect to the total weight of composition P.

The dimension of the diffracting pigment may be, for example, in the range of about 5 μm to about 200 μm, and preferably in the range of about 5 μm to about 30 μm.

The thickness of the particles of diffracting pigment may be about 3 μm or less, preferably about 2 μm, for example, of the order of about 1 μm.

Reflective Particles

The composition, for example, may include reflective particles, in particular flakes, which may or may not be magnetic.

The term “reflective particles” means particles in which the size, structure, the thickness of the layer or layers which constitute them, and physical or chemical nature and surface quality allows them to reflect incident light. The reflection may, if appropriate, be sufficiently intense to create, on the surface of the composition or mixture when applied to the surface to be made up, highlights which are visible to the naked eye, i.e. points which are more luminous which contrast with their environment and appear to shine.

The reflective particles may be selected so that they do not significantly alter the color effect generated by coloring agents that are associated therewith, and particularly, to optimize this effect as regards color yield. Particularly, they may possess a yellow, pink, red, bronze, orangey, brown, gold, and/or coppery color or glint.

The reflective particles may be present in the composition in an amount of about 0.5% to about 60% with respect to the total weight of composition P, in particular in an amount of about 1% to about 30% by weight, and more particularly in an amount of about 3% to about 10% by weight.

The particles may have different forms, in particular the form of platelets, or they may be globular, in particular spherical.

Regardless of their form, the reflective particles may or may not have a multi-layered structure and, with a multi-layered structure, for example, they may have at least one layer of uniform thickness, in particular of a reflective material.

When the reflective particles do not have a multi-layered structure they may, for example, be composed of metal oxides, in particular oxides of titanium or iron obtained by synthesis.

When the reflective particles have a multi-layered structure, these may, for example, include a natural or synthetic substrate, in particular a synthetic substrate that is at least partially coated with at least one layer of a reflective material, and in particular at least one metal or metallic material. The substrate may be an organic and/or inorganic mono-material or multi-material. More particularly, the substrate may be selected from, for example, glasses, ceramics, graphite, metal oxides, aluminas, silicas, silicates, in particular aluminosilicates and borosilicates, synthetic mica, and mixtures thereof.

The reflective material may include a layer of metal or a metallic material.

Reflective particles may include those described in Japanese patent documents JP A 09188830, JP A 10158450, JP A 10158541, JP A 07258460, and JP A 05017710, each of which are incorporated herein by reference in their entirety.

Further examples of reflective particles including a mineral substrate coated with a layer of metal are particles including a borosilicate substrate coated with silver.

Particles with a glass substrate coated with silver in the form of platelets are sold under the trade name MICROGLASS METASHNE REFSX 2025 PS by TOYAL. Particles with a glass substrate coated with a nickel/chromium/molybdenum alloy are sold under the trade name CRYSTAL STAR GF 550, GF 2525 by the same supplier.

Regardless of their form, the reflective particles may also be selected from particles with a synthetic substrate at least partially coated with at least one layer of at least one metallic material, in particular a metal oxide selected, for example, from oxides of titanium, in particular TiO₂, of iron, in particular Fe₂O₃, of tin, of chromium, of barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures and alloys thereof.

Examples of such particles may include a synthetic mica substrate coated with titanium dioxide, or particles of glass coated either with brown iron oxide, titanium oxide, tin oxide or a mixture thereof, such as those sold under the trade name REFLECKS® by ENGELHARD.

The composition of the invention may include at least one nacre, which may or may not be magnetic.

Nacres

The term “nacre” means colored particles of any form, iridescent or otherwise, in particular those produced in the shells of certain mollusks or synthesized and which have a color effect produced by optical interference.

The nacres may be selected from nacreous pigments such as mica titanium coated with an iron oxide, mica coated with bismuth oxychloride, mica titanium coated with chromium oxide, mica titanium coated with an organic colorant in particular of the type mentioned above, and nacreous pigments based on bismuth oxychloride. They may also be mica particles having surfaces superimposed with at least two successive layers of metal oxides and/or organic coloring materials.

More particularly, the nacres may have a yellow, pink, red, bronze, orangey, brown, gold, and/or coppery glint.

Illustrative examples of nacres which may be introduced into the composition which may be mentioned are gold colored nacres sold by ENGELHARD under the trade name Brillant gold 212G (Timica), Gold 222C (Cloisonne), Sparkle gold (Timica), gold 4504 (Chromalite) and Monarch gold 233X(Cloisonne); bronze nacres sold by MERCK under the trade name Bronze fine (17384) (Colorana) and Bronze (17353) (Colorana) and by ENGELHARD under the trade name Super bronze (Cloisonne); orange nacres sold under the trade name ENGELHARD under the trade name Orange 363C (Cloisonne) and Orange MCR 101 (Cosmica) and by MERCK under the trade name Passion orange (Colorana) and Matte orange (17449) (Microna); nacres with a brown hue, in particular those sold by ENGELHARD under the trade name Nu-antique copper 340XB (Cloisonne) and Brown CL4509 (Chromalite); nacres with a copper glint in particular those sold by ENGELHARD under the trade name Copper 340A (Timica); nacres with a red glint, in particular those sold by MERCK under the trade name Sienna fine (17386) (Colorana); nacres with a yellow glint, in particular those sold by ENGELHARD under the trade name Yellow (4502) (Chromalite); nacres with a red hue and a gold glint sold by ENGELHARD under the trade name Sunstone G012 (Gemtone); pink nacres, in particular those sold by ENGELHARD under the trade name Tan opale G005 (Gemtone); black nacres with a gold glint in particular those sold by ENGELHARD under the trade name Nu antique bronze 240 AB (Timica), blue nacres in particular those sold by MERCK under the trade name Matte blue (17433) (Microna), white nacres with a silvery glint in particular those sold by MERCK under the trade name Xirona Silver and orangey pink gold-green nacres sold by MERCK under the trade name Indian summer (Xirona), and mixtures thereof.

Composition P may, for example, include at least one filler, which may or may not be magnetic.

Fillers

The term “filler” means particles of any form, which are insoluble in the medium of the composition, regardless of the temperature at which the composition is manufactured. A filler may act to modify the rheology or texture of the composition. The nature and quantity of particles may depend on the desired mechanical properties and the textures.

Examples of fillers may include talc, mica, silica, kaolin, sericite, powders of polyamide, polyolefins, for example polyethylene, polytetrafluoroethylene, polymethylmethacrylate, polyurethane, starch powders, and silicone resin beads.

Composition P may also include dyes, organic pigments or lakes.

Dyes, Organic Pigments and Lakes

The dyes may be liposoluble or hydrosoluble.

Examples of liposoluble dyes are Sudan red, DC Red 17, DC Green 6, b-carotene, soya oil, Sudan brown, DC Yellow 11, DC Violet 2, DC orange 5, quinoline yellow.

Examples of hydrosoluble dyes are beetroot juice and methylene blue.

As an example, the dyes may represent about 0.1% to about 20% of the weight of composition P, or even about 0.1% to about 6%, when present.

The organic lakes or pigments may be selected, for example, from the materials below and their mixtures:

cochineal red;

organic pigments of azo, anthraquinone, indigo, xanthene, pyrene, quinoline dyes, triphenylmethane, and fluorane;

organic lakes or insoluble salts of sodium, potassium, calcium, barium, aluminum, zirconium, strontium, titanium, acid dyes such as azo, anthraquinone, indigo, xanthene, pyrene, quinoline dyes, triphenylmethane and fluorane dyes, the dyes possibly including at least one carboxylic or sulfonic acid group.

Organic pigments which may be mentioned are those known by the following denominations: D&C Blue No 4, D&C Brown No 1, D&C Green No 5, D&C Green No 6, D&C Orange No 4, D&C Orange No 5, D&C Orange No 10, D&C Orange No 11, D&C Red No 6, D&C Red No 7, D&C Red No 17, D&C Red No 21, D&C Red No 22, D&C Red No 27, D&C Red No 28, D&C Red No 30, D&C Red No 31, D&C Red No 33, D&C Red No 34, D&C Red No 36, D&C Violet No 2, D&C Yellow No 7, D&C Yellow No 8, D&C Yellow No 10, D&C Yellow No 11, FD&C Blue No 1, FD&C Green No 3, FD&C Red No 40, FD&C Yellow No 5, FD&C Yellow No 6.

The organic dye may include, for example, an organic lake supported by an organic support such as colophane or aluminum benzoate.

Particular examples of organic lakes may include those known by the following denominations: D&C Red No 2 Aluminum lake, D&C Red No 3 Aluminum lake, D&C Red No 4 Aluminum lake, D&C Red No 6 Aluminum lake, D&C Red No 6 Barium lake, D&C Red No 6 Barium/Strontium lake, D&C Red No 6 Strontium lake, D&C Red No 6 Potassium lake, D&C Red No 7 Aluminum lake, D&C Red No 7 Barium lake, D&C Red No 7 Calcium lake, D&C Red No 7 Calcium/Strontium lake, D&C Red No 7 Zirconium lake, D&C Red No 8 Sodium lake, D&C Red No 9 Aluminum lake, D&C Red No 9 Barium lake, D&C Red No 9 Barium/Strontium lake, D&C Red No 9 Zirconium lake, D&C Red No 10 Sodium lake, D&C Red No 19 Aluminum lake, D&C Red No 19 Barium lake, D&C Red No 19 Zirconium lake, D&C Red No 21 Aluminum lake, D&C Red No 21 Zirconium lake, D&C Red No 22 Aluminum lake, D&C Red No 27 Aluminum lake, D&C Red No 27 Aluminum/Titanium/Zirconium lake, D&C Red No 27 Barium lake, D&C Red No 27 Calcium lake, D&C Red No 27 Zirconium lake, D&C Red No 28 Aluminum lake, D&C Red No 30 lake, D&C Red No 31 Calcium lake, D&C Red No 33 Aluminum lake, D&C Red No 34 Calcium lake, D&C Red No 36 lake, D&C Red No 40 Aluminum lake, D&C Blue No 1 Aluminum lake, D&C Green No 3 Aluminum lake, D&C Orange No 4 Aluminum lake, D&C Orange No 5 Aluminum lake, D&C Orange No 5 Zirconium lake, D&C Orange No 10 Aluminum lake, D&C Orange No 17 Barium lake, D&C Yellow No 5 Aluminum lake, D&C Yellow No 5 Zirconium lake, D&C Yellow No 6 Aluminum lake, D&C Yellow No 7 Zirconium lake, D&C Yellow No 10 Aluminum lake, FD&C Blue No 1 Aluminum lake, FD&C Red No 4 Aluminum lake, FD&C Red No 40 Aluminum lake, FD&C Yellow No 5 Aluminum lake, FD&C Yellow No 6 Aluminum lake.

The chemical materials corresponding to each of the organic dyes mentioned above are mentioned in the work “International Cosmetic Ingredient Dictionary and Handbook”, 1997 edition, pages 371 to 386 and 524 to 528, published by “The Cosmetic, Toiletry and Fragrance Association” the contents of which are hereby incorporated by reference.

Composition P may include a composite pigment.

Composite Pigments

The composite pigment may be composed of particles including:

a magnetic or non-magnetic inorganic core;

at least one at least partial coating of at least one organic coloring material.

At least one binder may advantageously contribute to fixing the organic coloring material on the inorganic core.

The composite pigment particles may have various forms. The particles may be in the form of platelets or they may be globular, in particular spherical, and may be hollow or solid. The term “in the form of platelets” designates particles for which the ratio of the largest dimension to the thickness is about 5 or more.

A composite pigment may, for example, have a specific surface area in the range of about 1 m2/g [square meters per gram] to about 1000 m2/g, in particular in the range from about 10 m2/g to about 600 m2/g, and in particular in the range from about 20 m2/g to about 400 m2/g. The specific surface area is the value measured by the BET method.

The inorganic core of the composite pigment may have any form suitable for binding particles of organic coloring material, for example, spherical, globular, granular, polyhedral, acicular, spindle-shaped, flattened into the form of a flake, a grain of rice, a scale, an d/or a combination of those forms.

The ratio of the largest dimension of the core to its smallest dimension may be in the range of about 1 to about 50.

The inorganic core may have a dimension in the range from about 1 nm [nanometers] to about 100 nm, or even in the range from about 5 nm to about 75 nm, for example in the range from about 10 nm to about 50 nm.

The inorganic core may be produced from a material selected from, for example, metal salts and metal oxides, in particular oxides of titanium, zirconium, cerium, zinc, iron, ferric blue, aluminum and chromium, aluminas, glasses, ceramics, graphite, silicas, silicates, in particular aluminosilicates and borosilicates, synthetic mica, and mixtures thereof.

Oxides of titanium, in particular TiO₂, of iron, in particular Fe₂O₃, of cerium, of zinc, and of aluminum, silicates, in particular aluminosilicates and borosilicates, are particularly suitable.

The inorganic core may have a specific surface area, measured by the BET method, included, for example, in the range from about 1 m2/g to about 1000 m2/g, preferably in the range from about 10 m2/g to about 600 m2/g, for example in the range from about 20 m2/g to about 400 m2/g.

The inorganic core may be colored if appropriate.

The organic coloring material may be as defined above.

The binder for the composite pigment may be any type provided that it allows the organic coloring material to adhere to the surface of the inorganic core.

The binder may be selected from, for example, silicone materials, polymeric, oligomeric or the like materials, and in particular from organosilanes, fluoroalkyl organosilanes and polysiloxanes, for example polymethyl-hydrogen-siloxane, as well as various coupling agents, such as coupling agents based on silanes, titanates, aluminates, zirconates, and mixtures thereof.

The composition may include a photochromic coloring material or a photochromic agent.

Photochromic Agents

In general, a photochromic coloring agent may be a coloring agent having the property of changing hue when it is illuminated by ultraviolet light and re-establishing its initial color when it is no longer illuminated by the light, or of changing from a non-colored state to a colored state and vice versa. In other words, such an agent has different hues depending on whether it is illuminated by light containing a certain amount of UV radiation, such as sunlight or artificial light.

Reference can be made to the examples of photochromic agents described in European patent EP A 1 410 786, the disclosure of which is incorporated herein by reference.

Thermochromic Agents

It is possible, for example, to use a thermochromic agent sold under reference number KROMAFAST YELLOW 5GX 02- by KROMACHEM LTD.

Other Coloring Agents

Composition P may also include piezochromic compounds, in particular, tribochromic or solvatochromic compounds.

Other Components

Composition P includes a physiologically acceptable medium. The term “physiologically acceptable medium” means a non toxic medium which is capable of being applied to the skin, hair and nails, or the lips of human beings. The physiologically acceptable medium is generally adapted to the nature of the surface on which the composition is to be applied and to the form in which the composition is packaged.

Composition P may include ingredients other than those described above, in particular at least one solvent, a fatty phase, a film-forming polymer and/or a dermatological or cosmetically active ingredient, in particular as a function of the form.

Solvents

Composition P may include at least one aqueous or organic solvent, in particular, at least one volatile organic solvent, especially a volatile organic oil.

Within the context of the present invention, the term “volatile solvent” means a solvent which is liquid at ambient temperature, having a non-zero vapor pressure, at ambient temperature and atmospheric pressure, in particular having a vapor pressure of about 0.13 Pa [Pascals] to about 40000 Pa (about 10⁻³ mm Hg to about 300 mm Hg), preferably about 1.3 Pa to about 13000 Pa (about 0.01 mm Hg to about 100 mm Hg), preferably about 1.3 Pa to about 1300 Pa (about 0.01 mm Hg to about 10 mm Hg).

When composition P includes one or more organic solvents, the solvents may be present in an amount of about 0.1% to about 99% with respect to the total weight of the composition in question.

In general, the quantity of solvent(s), in particular organic solvent, depends on the nature of the surface onto which the composition is intended to be applied.

Composition P may include at least one volatile solvent constituted by a volatile oil.

The oil may be a silicone oil or a hydrocarbon oil, or it may include a mixture of the oils.

In the context of the present invention, the term “silicone oil” means an oil including at least one atom of silicon, in particular at least one Si—O group.

The term “hydrocarbon oil” means an oil principally containing atoms of hydrogen and carbon, and possibly also atoms of oxygen, nitrogen, sulfur, and/or phosphorus.

The volatile hydrocarbon oils may be selected from hydrocarbon oils containing 8 to 16 carbon atoms, in particular C8-C16 branched alkanes (also termed isoparaffins) such as isododecane (also termed 2,2,4,4,6-pentamethylheptane), isodecane, isohexadecane and, for example, oils sold under the trade names ISOPARS® or PERMETHYLS®.

Volatile oils which may also be used are volatile silicones, such as volatile linear or cyclic silicone oils, in particular those with a viscosity ≦8 centistokes (8×10⁻⁶ m²/s), especially containing 2 to 10 silicon atoms, in particular 2 to 7 silicon atoms, the silicones optionally including alkyl or alkoxy groups containing 1 to 10 carbon atoms. Volatile silicone oils which may be used in the invention which may be mentioned are dimethicones with a viscosity of about 5 cSt [centistokes] to about 6 cSt, octamethyl cycloetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

It is also possible to use fluorinated volatile oils such as nonafluoromethoxybutane or perfluoromethylcyclopentane, and mixtures thereof.

The composition may include in the range of about 0.01% to about 95% by weight of volatile oil with respect to the total composition weight, preferably in the range of about 1% to about 75% by weight.

Composition P may include at least one organic solvent selected from the following list:

ketones which are liquid at ambient temperature, such as methylethylketone, methylisobutylketone, diisobutylketone, isophorone, cyclohexanone, acetone;

alcohols which are liquid at ambient temperature, such as ethanol, isopropanol, diacetone alcohol, 2-butoxyethanol, cyclohexanol;

glycols which are liquid at ambient temperature, such as ethylene glycol, propylene glycol, pentylene glycol, glycerol;

propylene glycol ethers which are liquid at ambient temperature, such as propylene glycol monomethyl ether, propylene glycol monomethyl ether acetate, dipropylene glycol mono n-butyl ether;

short chain esters (containing 3 to 8 carbon atoms in total), such as ethyl acetate, methyl acetate, propyl acetate, n-butyl acetate, isopentyl acetate;

alkanes which are liquid at ambient temperature, such as decane, heptane, dodecane, cyclohexane.

Composition P may also include water or a mixture of water and hydrophilic organic solvents routinely used in cosmetics such as alcohols and especially linear or branched lower mono-alcohols containing 2 to 5 carbon atoms, such as ethanol, isopropanol or n-propanol, polyols such as glycerin, diglycerin, propylene glycol, sorbitol, penthylene glycol, polyethylene glycols. Composition P may also contain C2 ethers and hydrophilic C2-C4 aldehydes. The water or mixture of water and hydrophilic organic solvents may be present in the composition in an amount which is, for example, from about 0 to about 90%, in particular about 0.1% to about 90% by weight, preferably about 0 to about 60% by weight, in particular about 0.1% to about 60% by weight with respect to the total composition weight.

Fatty Phase

Composition P, for example, when intended to be applied to the lips, may include a fatty phase and in particular at least one fat which is liquid at ambient temperature (about 25° C.) and/or a fat which is solid at ambient temperature, such as waxes, pasty fats, gums, and mixtures thereof. Further, the fatty phase may contain lipophilic organic solvents.

Composition P may, for example, have a continuous fatty phase, which may contain less than about 5% water, in particular less than about 1% of water with respect to its total weight and in particular it may be in the anhydrous form.

Fats which are liquid at ambient temperature, often termed “oils” may include: vegetable hydrocarbon oils such as liquid triglycerides of fatty acids containing 4 to 10 carbon atoms, such as heptanoic or octanoic acid triglycerides, or sunflower seed, corn, soya, grapeseed, sesame, apricot, macadamia, castor or avocado oils, triglycerides of caprylic/capric acid, jojoba oil, shea butter oil, lanolin, acetylated lanolin, linear or branched hydrocarbons of mineral or synthetic origin such as paraffin oils and derivatives thereof, Vaseline, polydecenes, hydrogenated polyisobutene such as Parleam; synthesized esters and ethers such as those from fatty acids, such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, decanoates of fatty alcohols; isononyl isonanoate, isopropyl lanolate, tridecyl trimellilate, diisostearyl malate; polyol esters such as propylene glycol dioctanoate, neopentylglycol diheptanoate, diethylene glycol diisononanoate; pentaeythritol esters; fatty alcohols containing 12 to 26 carbon atoms, such as octyldodecanol, 2-butyloctanol, 2-hexyldecanol, 2-undecylpentadecanol, oleic alcohol; partially fluorinated hydrocarbon and/or silicone oils; silicone oils such as polymethylsiloxanes (PDMS), volatile or otherwise, linear or cyclic, liquid or pasty at ambient temperature such as cyclomethicones, dimethicones, optionally including a phenyl group, such as phenyltrimethicones, phenyltrimethylsiloxydiphenylsiloxanes, diphenylmethyldimethyl-trisiloxanes, diphenyldimethicones, phenyl dimethicones, polymethylphenyl siloxanes; and mixtures thereof. The oils may be present in an amount of about 0.01% to about 90%, preferably about 0.1% to about 85% by weight with respect to the total composition weight.

The presence of an oily phase may provide gloss and, for example, a refractive index in the range of about 1.47 to about 1.51, preferably in the range of about 1.48 to about 1.50. The refractive index is measured at ambient temperature (about 25° C.) using a refractometer.

Composition P may include a pasty fat, a wax, or a gum.

Pasty fats are generally hydrocarbon compounds with a melting point in the range of about 25° C. to about 60° C., preferably in the range of about 30° C. to about 45° C., and/or a hardness in the range of about 0.001 MPa to about 0.5 MPa, preferably in the range of about 0.005 MPa to about 0.4 MPa, such as lanolins and derivatives thereof.

The waxes may be solid at ambient temperature (about 25° C.), with a reversible solid/liquid change of state, having a melting temperature of more than about 30° C. which may be up to about 200° C., with a hardness of more than about 0.5 MPa, and having an anisotropic crystalline organization in the solid state. In particular, the waxes may have a melting point of more than about 25° C., preferably more than about 45° C. The waxes may be hydrocarbon, fluorinated, and/or silicone waxes and be of vegetable, mineral, animal, and/or synthetic origin. Waxes which may be used include, for example, beeswax, carnauba wax or candellila wax, paraffin, microcrystalline waxes, ceresine or ozokerite; synthetic waxes such as polyethylene waxes or Fischer-Tropsch waxes, silicone waxes such as alkyl or alkoxy-dimethicone waxes containing 16 to 45 carbon atoms. The composition may contain 0 to about 50% by weight of waxes relative to the total composition weight, or even about 1% to about 30% by weight.

Gums which may be used are generally polydimethylsiloxanes (PDMS) with a high molecular weight, cellulose gums or polysaccharides.

Film-Forming Polymers

Composition P may also, for example, include a film-forming polymer, in particular in the case of a mascara or a nail varnish. The term “film-forming polymer” designates a polymer which may, by itself or in the presence of an auxiliary film-forming agent, form a continuous film which adheres to a surface, in particular to keratinous materials.

Examples of film-forming polymers which may be used in composition P include, for example, synthetic polymers, of the radical or polycondensate type, polymers of natural origin such as nitrocellulose or cellulose esters, and mixtures thereof.

Radical type film-forming polymers may be vinyl polymers or copolymers, in particular acrylic polymers.

Vinyl film-forming polymers may result from polymerizing monomers with an ethylenically unsaturated bond containing at least one acid group and/or esters of the acid monomers and/or amides of the acid monomers like unsaturated a,b-ethylenic carboxylic acids such as acrylic acid, methacrylic acid, crotonic acid, maleic acid, or itaconic acid.

Vinyl film-forming polymers may also be the result of homopolymerization or copolymerization of monomers selected from vinyl esters such as vinyl acetate, vinyl neodecanoate, vinyl pivalate, vinyl benzoate or vinyl t-butyl benzoate and styrene monomers such as styrene and alpha-methyl styrene.

Film-forming polycondensates may include, for example, polyurethanes, polyesters, polyester amides, polyamides and polyureas.

Polymers of natural origin which may be modified may be selected from shellac resin, sandarac gum, dammar resins, elemi gums, copal gums, cellulose polymers such as nitrocellulose, ethyl cellulose or nitrocellulose esters selected, for example, from cellulose acetate, cellulose acetobutyrate and cellulose acetopropionate, and mixtures thereof.

The film-forming polymer may be present in the form of solid particles in aqueous or oily dispersion, generally known as latexes or pseudolatexes. The film-forming polymer may include one or more stable dispersions of particles of generally spherical polymers of one or more polymers in a physiologically acceptable liquid fatty phase. The dispersions are generally termed NAD (non aqueous dispersions) of polymers as opposed to latexes, which are aqueous dispersions of a polymer. The dispersions may be in the form of nanoparticles of polymers in stable dispersion in the fatty phase. The nanoparticles preferably have a size in the range of about 5 nm to about 600 nm. Techniques for preparing such dispersions are well known.

Examples of aqueous film-forming polymer dispersions which may be used are acrylic dispersions sold under the trade name NEOCRYL XK-90®, NEOCRYL A-1070®, NEOCRYL A-1090®, NEOCRYL BT-62®, NEOCRYL A-1079®, NEOCRYL A-523® by AVECIA-NEORESINS, DOW LATEX 432® by DOW CHEMICAL, DAITOSOL 5000 AD® by DAITO KASEI KOGYO; or aqueous dispersions of polyurethane sold under the trade name NEOREZ R-981®, NEOREZ R-974® by AVECIA-NEORESINS, AVALURE UR-405®, AVALURE UR-410®, AVALURE UR-425®, AVALURE UR -450®, SANCURE 875®, SANCURE 861®, SANCURE 878®, SANCURE 2060® by GOODRICH, IMPRANIL 85® from BAYER, AQUAMERE H-1511® by HYDROMER; sulfopolyesters sold under the trade name Eastman AQ by Eastman Chemical Products.

Composition P may also include an auxiliary film-forming agent encouraging the formation of a film with the film-forming polymer.

Active Ingredients

Composition P may include at least one cosmetically or dermatologically active ingredient. Cosmetically, dermatologically, hygienically or pharmaceutically active ingredients which may be used include, for example, moisturizers (polyol such as glycerin), vitamins, (C, A, E, F, B or PP), essential fatty acids, essential oils, ceramids, sphingolipids, liposoluble sunscreens or sunscreens in the form of nanoparticles, specific active ingredients for the treatment of skin (protective agents, antibacterials, anti-wrinkle agents, etc), self-tanning agents. The active ingredients may, for example, be used in concentrations of 0 to about 20% and especially about 0.001% to about 15% with respect to the total composition weight.

Composition P may also contain ingredients which are routinely used in cosmetics, such as thickeners, surfactants, oligo elements, moisturizers, softeners, sequestrating agents, fragrances, alkalizing or acidifying agents, preservatives, anti-oxidants, UV screens, colorants, or mixtures thereof.

Depending on the envisaged type of application, composition P may include constituents conventionally used in the fields under consideration which are present in a quantity appropriate for the desired form.

The characteristics of the various examples may be combined together in variations (not shown).

If necessary, a motif may be formed by applying a magnetic field to a first layer deposited on a surface to be made up. After drying, the first layer, may be removably covered by a second layer or structure which is held on the first layer by the magnetic interaction existing with the first layer.

In a variation (not shown), a magnetic mascara is applied to the eyelashes. A magnetic powder is dusted over the lashes. Magnetic attraction holds the particles on the lashes.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Unless otherwise specified, the expression “including a” should be understood as being synonymous with “including at least one.”

What is claimed is:

1. A method of making up or decorating keratinous materials, comprising:
i) applying, to the keratinous materials, a first layer of a first composition or a first structure having magnetic properties;
ii) applying, to at least a portion of the first layer or first structure, a second layer of a second composition or a second structure interacting magnetically with the first layer or with the first structure;
at least one of the first and second structures comprising dispersed magnetic bodies in a non-magnetic medium.

2. A method according to claim 1, wherein the keratinous materials is selected from among skin, nails, and eyelashes.

3. A method according to claim 1, in which the magnetic interaction ensures that the second layer or second structure is retained on the first layer or first structure.

4. A method according to claim 1, in which a composition comprising dispersed magnetic bodies in a medium is applied during step ii), the magnetic interaction contributing to orientating and/or displacing at least a portion of the magnetic bodies present in the composition.

5. A method according to claim 1, in which a first layer of a first composition having magnetic properties is applied in step i), and a structure at least partially covering the first layer of composition which magnetically interacts with the latter is applied in step ii).

6. A method according to claim 4, in which the first composition contains at least one volatile solvent and in which application of the structure in step ii) is carried out after drying the first composition.

7. A method according to claim 5, in which the structure applied in step ii) is a false nail.

8. A method according to claim 5, in which the structure applied in step ii) is a false tattoo, film or patch, or comprises a plurality of decorative elements such as a powder, fibers, or flakes.

9. A method according to claim 1, in which the first structure having magnetic properties is applied in step i), and
a deposit of a composition is formed over all or part thereof, or
a second structure is applied, the deposit and the second structure interacting magnetically with the first structure.

10. A method according to claim 9, in which the first structure is adhesive.

11. A method according to claim 9, in which the first structure comprises a flexible film.

12. A method according to claim 1, in which the second layer of the composition is applied in step ii), the second layer comprising magnetic bodies capable of being orientated under the effect of a magnetic interaction with the first layer or the first structure.

13. A method according to claim 2, in which the first composition contains at least one volatile solvent and in which application of the composition in step i) is carried out after drying the first composition.

14. A method according to claim 12, in which the first layer or the first structure forms a motif.

15. A method according to claim 12, further comprising applying the first layer manually or using an inkjet printer.

16. A method according to claim 12, further comprising applying the first layer by transfer.

17. A method according to claim 1, in which the first layer or structure is permanently magnetized.

18. A method according to claim 1, in which the second layer or structure is permanently magnetized.

19. A method according to claim 1, in which the first composition is a nail varnish.

20. A method according to claim 1, in which the first composition is a mascara.

21. A hand makeup kit, comprising:
at least one article having magnetic properties; and
a form which is suitable for fixing the at least one article to the nail by magnetic attraction.

22. A makeup kit comprising:
at least one receptacle containing a composition having magnetic properties;
at least one magnetic structure configured to be fixed by magnetic attraction to a surface on which a layer of the composition contained in the receptacle has been deposited.

23. A kit according to claim 22, in which the magnetic structure is a false nail.

24. A kit according to claim 22, in which the magnetic structure comprises a powder, fiber, or flake.

25. A kit according to claim 22, in which the magnetic structure is a film, a beauty spot, or a false tattoo.

26. A kit according to claim 22, in which the composition comprises at least one volatile solvent.

27. A kit according to claim 22, in which the composition comprises at least one film-forming polymer.

28. A makeup kit comprising:
a first receptacle containing a first composition containing bodies comprising metallic iron;
a second receptacle containing a second composition comprising magnetic bodies, the second composition configured to, when it is applied in the form of a layer above or below a layer formed by the first composition, interact magnetically with the first composition;
the magnetic bodies of at least one of the first and second compositions being permanently magnetized.
